(19) United States
(12) Patent Application Publication
(10) Pub. No.: US 2023/0338168 A1
(43) Pub. Date: Oct. 26, 2023

(54) PROSTHESIS COSMETIC AND METHOD FOR PRODUCING SAME

(71) Applicant: OTTO BOCK HEALTHCARE PRODUCTS GMBH, Wien (AT)

(72) Inventors: Sonja WAGNER, Wien (AT); Walter LUNZER, Wien (AT); Alice SCHERB, Stockerau (AT)

(73) Assignee: OTTO BOCK HEALTHCARE PRODUCTS GMBH, Wien (AT)

(21) Appl. No.: 17/916,014

(22) PCT Filed: Apr. 1, 2021
(86) PCT No.: PCT/EP2021/058676

§ 371 Date: Sep. 29, 2022

(30) Foreign Application Priority Data
Apr. 3, 2020 (DE) 10 2020 109 337.3

(51) Int. Cl. A61F 2/60; A61F 2/50; B32B 5/18; B32B 5/02; B32B 5/24; B32B 7/12

(52) U.S. Cl. CPC A61F 2/60; A61F 2/5044; B32B 5/18; B32B 5/02; B32B 5/245; B32B 7/12; A61F 2002/607; A61F 2002/5001; A61F 2002/5081; A61F 2002/5007; B32B 2535/00

(57) ABSTRACT

A prosthesis cosmetic having a main part made of a foam material and/or composite fiber material and a cavity formed in the main part for receiving a prosthesis component, wherein the main part has an outside facing away from the cavity, on which outside a textile layer is arranged, an adhesive being applied on the inside of the textile layer facing the main part and the textile layer being adhered or laminated on the outside of the main part.

### CROSS REFERENCE TO RELATED APPLICATIONS

This is a national phase application of International Application No. PCT/EP2021/058676, filed 1 Apr. 2021, which claims the benefit of German Patent Application No. 10 2020 109 337.3, filed 3 Apr. 2020, the disclosures of which are incorporated herein, in their entireties, by this reference.

### TECHNICAL FIELD

The invention relates to a prosthesis cosmetic with a base body made of a foam material and/or fiber composite material and with a cavity formed in the body for receiving a prosthesis component, the base body having an outer side which faces away from the cavity and on which a textile layer is arranged, and a method for producing same.

### BACKGROUND

Prosthesis cosmetics are used to cover a prosthesis component, on the one hand with the aim of achieving the most natural appearance possible for the replaced limb, and on the other hand for protecting the prosthesis component from external influences. In addition, prosthesis cosmetics can protect the surroundings from direct contact with the prosthesis component, by making available a padding for the prosthesis component.

The manufacture of individual prosthesis cosmetics is highly complex, and therefore the usual procedure when producing a prosthesis cosmetic for a specific prosthesis component entails adapting a base body, which has a cavity for receiving the prosthesis component, to the respective person through removal of material. As a rule, it is sought to produce the prosthesis component with a shape that corresponds to the intact side. The base body is generally made of a light, voluminous material, for example a foam material, which is processed by the relevant specialist personnel. The shape of the outer contour is worked out by grinding or by other cutting processes.

The foam material or the fiber composite material can be designed to be elastic, in order to absorb impacts and to cushion the prosthesis component with respect to the environment. However, as a result of the processing with the cutting methods that are deemed suitable in each case, the outer surface of the base body becomes rough. Before processing, the foamed base body has a smooth, often also closed surface, whereas the finished and shaped base body has a rough outer surface without the characteristic finish or without what is called an integral skin. A rough surface of this kind is disadvantageous during use, because clothing sticks to it or rubs against it. This makes it difficult to put on or take off clothes, and, what is more, the clothes catch on the rough surface, and therefore items of clothing that have slipped upward do not slide back down again.

Textile covers are known from the prior art and are pulled in the form of a hose over the surface of the base body. WO 2013/079159 A1 discloses a prosthesis cosmetic for a lower extremity, which prosthesis cosmetic is formed from several components or portions. A distal portion can initially have an open cross section over part of its longitudinal extent. A covering made of a highly elastic material, for example a highly elastic textile, can be arranged on the outer side of the portions.

An alternative provides that an individualized base body is covered with a stocking, which is permanently attached to the base body at its proximal end. A disadvantage of this is that it is very time-consuming and furthermore restricts the field of use of the prosthesis cosmetic, since it is permanently attached to the prosthesis component.

### SUMMARY

The object of the present invention is to make available a prosthesis cosmetic and a method for producing same, with which the disadvantages of the prior art are avoided. According to the invention, this object is achieved by a prosthesis cosmetic having the features of the main claim and by a method having the features of the additional independent claim. Advantageous embodiments and refinements of the invention are disclosed in the subclaims, the description and the figures.

In the prosthesis cosmetic with a base body made of a foam material and/or fiber composite material and with a cavity formed in the base body for receiving a prosthesis component, the base body having an outer side which faces away from the cavity and on which a textile layer is arranged, provision is made that an adhesive is applied to the inner side of the textile layer facing the base body, and the textile layer is adhesively bonded to the outer side of the base body. The textile layer adhesively bonded to the outer side of the base body serves to seal the roughened foam material and/or the fiber composite material in particular and acts like a subsequently applied integral skin, which is otherwise only obtained in the primary forming of a foam material and/or fiber composite material. After the processing operation, the textile layer gives the base body a smooth surface, which makes putting on items of clothing easier for the user to handle. The adhesive can be applied to the inner side of the textile layer and/or the outer side of the base body before the textile layer is fitted, in particular also to a flat basic blank before the latter is sewn together to form a tube or cone.

The adhesive can be designed to be thermally activatable, which facilitates application of the textile layer, since there is little or no adhesive effect or tack at room temperature. In the production of the prosthesis cosmetic, it is only necessary to place the textile layer onto the base body and, in particular, to activate it thermally, which ensures that the textile layer is glued on over a large surface area and is securely attached to the base body.

The textile layer can be formed from a tube or a flat blank. The tube has a closed cross section and is coated or lined on the inner side with the thermally activatable adhesive. This can be done, for example, by first turning the surface intended as the inner side outward, that is to say turning the tube “wrong side out”, and then coating it with the adhesive in the intended regions. To place the tube onto the fully formed base body, the tube can either be unrolled on the outer side of the base body or pulled over the base body. As an alternative or in addition, an adhesive or an adhesive component is applied to the outer side of the base body and brings about the adhesive bonding upon contact with the textile layer or thereafter. The tube, or a tube formed from a blank, can have, at its upper end, a shortened or turned edge, which can be folded over for fastening to the base body or already has an inwardly folded edge or an inner textile strip, such that an improved appearance of the finished product can be obtained with less labor.

As an alternative to the design of the textile layer from a tube with a closed cross section, it can be formed from a flat blank, the flat blank being coated on one side with a preferably thermally activatable adhesive and being firmly glued to the outer side of the base body. The blank can be closed via a seam, for example a chain stitch seam, such that a hose-like structure is created which is pulled over the base body and can be permanently fixed to the base body upon activation of the adhesive.

The textile layer and/or the base body can be composed of a flexible, in particular elastic material, such that, in the case of a flexible material, the textile layer can be easily applied to the individually manufactured surface of the base body. A non-elastic textile layer can also be used. In this case, the shape is ensured by means of darts, for example. The flexibility makes it possible to ensure that the textile layer is placed on the base body without creases. In the case of an elastic configuration of the textile layer, it is possible to stretch the latter when applying it to the base body, such that there is an automatic adaptation to the contour of the base body. In addition, a pressure is applied to the adhesive layer, which permits secure adhesion when the adhesive is activated. In the case of an elastic configuration of the base body, the latter can exert a cushioning function. At the same time, it is possible to equip the base body with an open cross section, such that the base body, together with the applied textile, can be bent open and removed from the prosthesis component or easily placed on the prosthesis component. In one embodiment, the textile layer and/or the base body are formed from a pre-shaped, in particular non-elastic material.

To make it easier to put on and take off the prosthesis cosmetic, the base body has a slit with two mutually opposite end faces which, in one variant, bear against each other in the applied state. The slit can be arranged substantially oriented along a longitudinal extent of the prosthesis cosmetic and can run oriented radially in the direction of the cavity. In principle, other profiles of the slit and other orientations can also be formed which are adapted to the shapes of the base body. In addition to ease of opening, it is advantageous to have the best possible overlap and the smallest possible gap in the slit between the end faces, so as to afford the most natural possible appearance.

The textile layer can be glued onto the end faces in order, on the one hand, to achieve complete coverage on the outer side of the base body and, on the other hand, to achieve improved adhesion of the textile layer to the base body. The gluing to the end faces prevents the textile layer from coming loose, in particular at the exposed transition from the outer side to the end face. Coming loose is easiest in this region, and there is the greatest risk here of the textile detaching or fraying.

A fastener can be arranged on the textile layer, via which fastener the end faces are coupled to each other in the closed state of the fastener. The fastener can be designed, for example, as a zip fastener, a Velcro fastener, a hook fastener or a magnetic fastener and connects the two end faces directly to each other, thus preventing the end faces from moving away from each other. If a gap is present between the two end faces, said gap cannot grow larger after the fastener is closed. In principle, it is also possible for a magnetic fastener to be arranged within the textile layer.

A further development provides that the textile layer has, at the upper, proximal end and/or the lower, distal end of the textile layer, a pocket, a turn-back region or a receptacle for the proximal edge of the base body. The pocket or receptacle or the turn-back region can be formed by folding over and fixing a portion of a part of the textile layer or by sewing on or otherwise fixing, for example gluing or welding, a separate textile blank. In the assembled state, the base body is inserted in the pocket, the turn-back region or the receptacle. In a further development, the adhesive is applied at least in certain regions on that side of the turned-back part or of the attached textile blank facing the base body. Alternatively or in addition, adhesive is applied to the inner side of the base body, in particular in the region in which the turned-back part or the attached textile blank lies or is intended to lie. The textile layer is then glued to the base body both on the outer side of the base body and also partially on the inner side of the base body.

Through the application of a textile layer, it is possible to adapt the prosthesis cosmetic to the particular purpose of use and, in addition, to make functional improvements, for example by coating the textile. The coatings or finishes can be adapted to the particular purpose of use, for example by the textile having a water-repellent, flame-retardant and/or antimicrobial action.

The method for producing a prosthesis cosmetic, as has been described above, involves making available a base body which is made of a foam material and which is adapted to a prosthesis component to be covered and to a prosthesis wearer. An adhesive, in particular a thermally activatable adhesive, is applied to a textile layer, specifically on an inner side of the textile layer which, in the applied state of the textile layer, faces the base body of the prosthesis cosmetic. The textile layer is then arranged with its inner side on the outer side of the base body and glued. The gluing takes place, for example, by activation of the thermally activatable adhesive, that is to say by the delivery of heat. Alternatively, the gluing can also be carried out by bringing into contact the adhesive and applying pressure alone. The textile layer is advantageously glued on with application not only of heat but also of pressure, in order to achieve a secure and crease-free contact of the entire textile layer on the outer side of the base body.

In order to exert a pressure on the textile layer and to press the textile layer onto the base body, it can be wrapped with a textile or a film or another flexible article, similarly to a pressure bandage, such that sufficient pressure is applied to press the textile onto the outer side of the base body.

The adhesive layer can be applied at least regionally on the inner side of the textile layer, the regions with the applied adhesive preferably being designed and arranged in such a way that the textile can be secured over a large surface area and as far as possible without creases. For this purpose, the adhesive layer can be applied in regions, in particular at points, over the entire contact surface on the base body. Even when using an adhesive that is not elastic, this means that a resilience is obtained on account of the flexibility and possibly elasticity of the textile. Alternatively, the adhesive layer can be applied over the whole surface area of the textile or over the entire region of the surface that rests on the base body.

A further development provides that the textile layer is formed as a one-piece tube or as a tube formed from a blank via a seam, in particular a chain stitch seam, and is pulled over the base body before gluing. The textile layer can initially be made available as a large-area basic blank, from which the final blank is manufactured, the latter being intended to be applied to the fully formed base body. Connecting two edges or sides of a blank facing away from each other via a chain stitch seam has the advantage that it can be opened again with one pull. After the textile or the tube has been pulled over the individually prepared, in particular ground base body, the textile is glued to the base body via the adhesive. If the adhesive is a thermally activatable adhesive, the adhesive is thermally activated for gluing, if necessary with application of compressive forces, for example by wrapping or also by means of clips or other pressure devices.

After the textile layer has been glued on, a slit can be made in the textile layer and, if appropriate, also in the base body. The slit in the textile layer preferably runs along the slit in the base body. If the blank of the textile is subsequently closed, for example by a seam, this seam is preferably oriented along an already existing slit within the base body, such that the slit can be easily opened together with the textile after the seam has been glued and opened.

Along the slit formed on the base body, the textile layer can be provided with a fastener, for example a zip fastener, which is secured to the textile, in particular sewn on, for which purpose the seam overhang or the seam allowance is suitable in particular. Further suitable regions are the inner side of the base body, the end faces, and/or the regions or outer surfaces framing the slit. If there is still material left after the fastener has been applied, this material can be glued to the end faces of the slit of the base body or otherwise cohesively bonded.

The invention also relates to a system composed of a base body made of a foam material and/or fiber composite material and of a textile coated with an adhesive, in particular a thermally activatable adhesive, as has been described above.

### BRIEF DESCRIPTION OF THE DRAWINGS

An exemplary embodiment of the invention is explained in more detail below with reference to the accompanying figures, in which:

FIG. 1 shows a base body of a prosthesis cosmetic, and a basic blank;

FIG. 2 shows a base body after shaping, and an end blank;

FIG. 3 shows an end blank sewn with a chain seam;

FIG. 4 shows a base body with an applied textile;

FIG. 5 shows a wrapped base body before heating;

FIG. 6 shows a prosthesis cosmetic with a glued-on textile with an opened chain seam and a slit in the base body;

FIG. 7 shows a fully assembled prosthesis cosmetic with adhesively bonded end faces and a fastener;

FIG. 8 shows a variant of the textile;

FIG. 9 shows a combination of base body and textile according to FIG. 8;

FIG. 10 shows a schematic representation of an assembly process; and

FIG. 11 shows a variant of the fastener.

### DETAILED DESCRIPTION

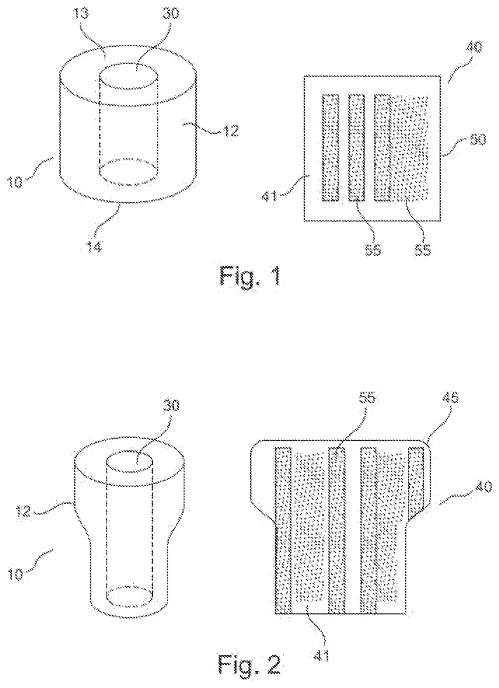

FIG. 1 shows a perspective view of a base body 10 of a prosthesis cosmetic and a basic blank 50 of a textile layer 40. In the exemplary embodiment shown, the base body 10 is cylindrical and has a central cavity 30, which substantially corresponds to the outer contour of the object to be received, i.e. the prosthesis component to be received (not shown). The cavity 30 can, for example, also be cylindrical in order to accommodate a lower-leg tube or the like. Alternatively, the cavity can have any desired shape in order to accommodate dampers, mechanical components, controls, sensors, energy stores and the like. In the exemplary embodiment shown, the cavity 30 extends from an upper side 13, which is located proximally in the applied state, to a lower side 14, which is oriented distally in the applied state. The base body 10 has a cylindrical outer side 12, which is located on that side of the base body 10 facing away from the cavity 30. The base body 10 is composed of a foam material and/or fiber composite material and can be dimensionally stable or also flexible, in particular elastic.

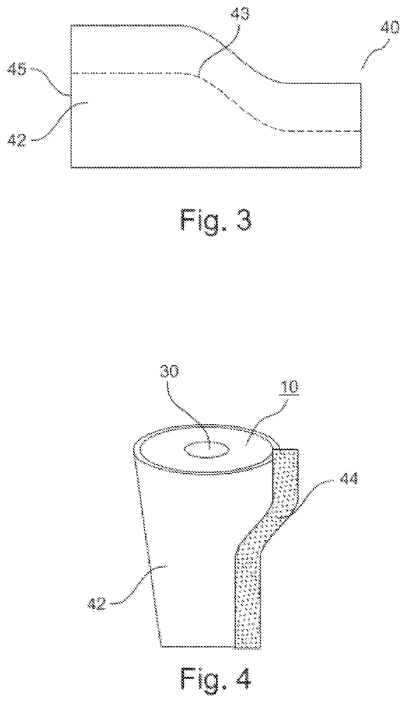

In addition to the base body 10, FIG. 1 shows the textile layer 40 arranged in the form of the basic blank 50. The basic blank 50 of the textile layer 40 is rectangular in the exemplary embodiment shown. Like every flat basic blank 50 of a textile, it has a top side and a bottom side which, when applied, become an inner side 41 and, facing away from the inner side 41 and thus also from the base body 10, an outer side 42, which is designated in FIG. 3. An adhesive 55 or an adhesive layer 55 is applied at least in certain regions on the inner side 41 of the textile layer 40. In the exemplary embodiment shown, only certain regions of the inner side 41 of the textile layer 40 are provided with the adhesive 55. The regions can have a comparatively large surface area and can be arranged in the form of strips. As an alternative or in addition, the regions with adhesive 55 can be formed as points and arranged regularly or irregularly on the inner side 41 of the textile layer 40. By coating the inner side 41 of the textile layer 40 with spaced-apart regions of adhesive 55 or with punctiform regions of adhesive, it is possible to use the textile or the textile layer 40 in a very versatile manner. In particular, in the case of an elastic configuration of the textile layer 40, it is possible to permit full-surface and smooth-surface contact even with complex surface contours of the base body 10 in the processed state.

The adhesive 55 is advantageously designed to be thermally activatable, but it can also have a slight tackiness at room temperature or at the usual temperatures present during processing, in order to make it easier to position the textile layer 40 on the base body 10.

FIG. 2 shows the next phase of processing. The previously cylindrical base body 10 is processed on its outer side 12 in such a way that it corresponds to the desired shape, for example it has an outer shape corresponding to the shape of the limb that a prosthesis component is intended to replace.

For example, the base body 10 can have the contour of a lower leg, while the cavity 30 is shaped in such a way that it can accommodate the prosthesis components of a lower leg prosthesis. The textile layer 40 has been processed from the previously rectangular shape into a blank 45 which has a contour adapted to the shape of the base body 10 that is to be enveloped. The adhesive layer 55 or regions with adhesive 55 is/are applied to the inner side 41 of the textile layer 40.

FIG. 3 shows the processed blank 45 in which a tubular covering has been formed via a seam 43, for example a chain stitch seam, and is open at the top and bottom such that the base body 10 can be inserted. The blank 45 then completely surrounds the base body 10.

The state of the processed base body 10 introduced into the tubular textile is shown in FIG. 4. The outer side 42 of the textile layer 40 is not provided with an adhesive layer but can, for example, be provided with a coating or finish that has a water-repellent, flame-retardant and/or antimicrobial action. Beyond the tubular region formed by the seam 43, where the base body 10 is not inserted, a seam overhang 44 is formed, which is left in place in the exemplary embodiment.

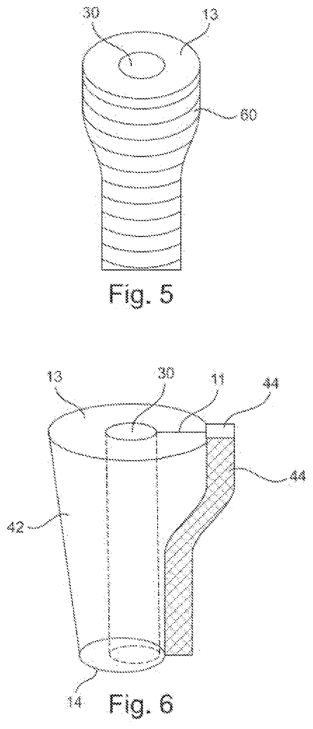

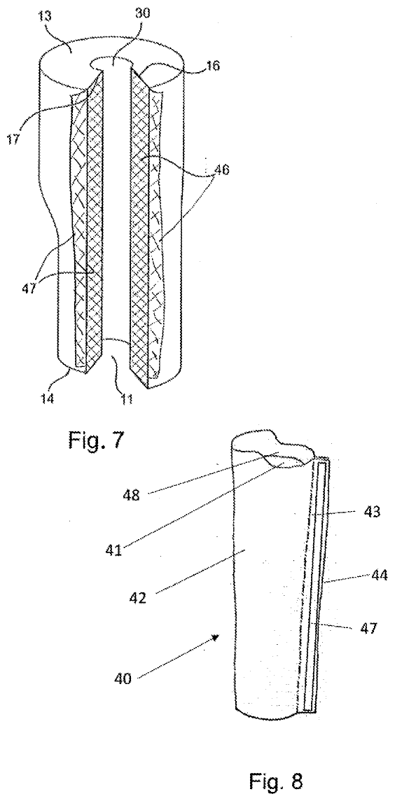

In a next processing step, which is shown in FIG. 5, the base body 10, together with the textile layer 40 on the outer side 42, is enveloped or sheathed by a textile band 60 or a film or is inserted into a cone-shaped sheath in order to exert a sufficient radial pressure on the textile 40. This ensures that the adhesive layer or the adhesive 55 on the inner side 41 of the textile 40 is pressed onto the outer side 12 of the base body 10. In this compressed state, the base body 10 is heated together with the textile 40 such that, by application of pressure and heat, the thermally activatable adhesive 55 is activated. This makes it possible to permanently glue the textile layer 40 to the outer side 12 of the base body 10. The textile 60 or the envelope is then removed. In the region of the joints of the textile 40, which are fixed to one another by the seam 43, a slit 11 can be made in the base body 10 after the seam material has been removed. This is shown in FIG. 6. The two seam overhangs 44 are located on both sides of the slit 11. By applying the slit 11, which protrudes from the radial outer side 12 into the cavity 30 and extends over the entire length, i.e. from the upper side 13 to the lower side 14, of the base body 10, it is possible to bend the base body 10 open. It is likewise possible to cover the end faces 16 and 17 in the base body 10, which are produced by the slit 11 and are shown in FIG. 7, with the textile material of the seam overhang 44. For this purpose, the seam overhang 44 can be provided with an adhesive and glued onto the respective end face 16, 17. Alternatively, the end faces 16, 17 can also be provided with adhesive and then covered with the textile material of the seam overhang 44.

On the mutually opposite end faces 16, 17 of the slit 11, a fastener 46, 47 can be arranged or formed on the respective outer side of the textile layer 40, for example as a Velcro fastener, for example with one end face 46 being equipped with a hook region and the opposite end face 47 with a fleece region. This makes it possible to open and close the finished prosthesis cosmetic along the slit 11, with closure being achieved via the form-fit elements. The prosthesis cosmetic can then be opened or closed repeatedly in order to remove it from the respective prosthesis component or to place it around the latter. Alternative fasteners are buckles, straps, clips, press studs, hooks, loops, buttons, magnets and the like. An additional or alternative closure concept in FIG. 7 involves fastener elements or parts of the fastener elements 46, 47 which are arranged on the outer side of the textile 40. For example, a right-hand fastener element 46 is arranged on what is the outer side of the textile 40 in the closed state. An opposite fastener element 47 is arranged on the textile on the opposite edge or outer side of the slit 11 so as to be foldable as a flap or strip of fabric. The fastener elements 46, 47 are then fixed to each other or on each other by means of suitable connecting elements, for example a Velcro fastener or magnets or other devices that act in a form-fitting or force-fitting manner.

FIG. 8 shows a variant of the textile layer 40, in which the textile layer 40 is shaped into a tubular body. The upper, proximal end and the lower, distal end of the textile layer 40 are open. The textile layer 40 is produced from a blank 45 and has a seam overhang 44 which is located on the other side of the seam 43 running in the proximal-distal direction. A turn-back region 48 is arranged or formed at the upper end of the textile layer 40. The turn-back region 48 can be designed as part of a flat blank 45 and then, after the tubular body is formed by the seam 43, or before this, can be turned over onto the inner side 41. This creates a pocket which is provided with an adhesive layer or an adhesive 55 on its respective inner side, such that surfaces of the flat blank 45 that are provided with adhesive lie opposite one another. The adhesive can be applied to the inner side 41 before the turn-back region 48 is turned over. At the upper edge of the tubular body, the turn-back region 48 can be held, for example, by a fixing seam, wherein the inner, downwardly protruding end of the turn-back region 48 remains open in order to receive the base body 10 there. As an alternative or in addition, fixing can take place at the sides, i.e. the medial and lateral ends, of the turn-back region 48.

Instead of being an integral part of the blank 45, the turn-back region 48 can be a separate piece of textile which is attached to the rest of the blank 45, for example sewn, welded or glued on. Once again, it is advantageous to apply adhesive over the entire surface or in certain regions on that side of the additional piece of textile that faces inward in the assembled state.

In both cases, the proximal end of the textile layer 40 is shaped as the proximal edge of the base body 10 is shaped. In particular, that side of the textile layer 40 facing the base body 10 is coated in certain regions, for example at points, with an adhesive, in particular with an adhesive that can be activated by heat. The points of adhesive are arranged spaced apart from one another.

On the outer side of the seam allowance 44, a fastener 47 or a fastener element is shown schematically which, in the applied and assembled state, rests or bears on the end face 17 of the slit 11. The fastener element 47 can be designed, for example, as a Velcro fastener or a magnetic fastener.

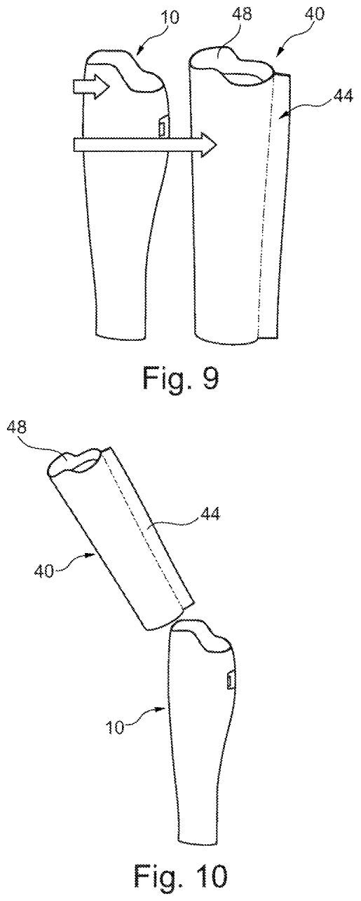

FIG. 9 shows the base body 10 and the textile layer 40 next to each other before they are assembled or brought together. The base body 10 is designed as a lower leg part of a prosthesis cosmetic and has an elevation at the proximal, upper end on the front and in the medial and lateral region, which elevation slopes down toward the rear or posterior region, where the calf muscle is located on the natural calf. The base body 10 is designed as a hollow body, at least the upper edge of the base body 10 being relatively narrow and leaving space for further technical prosthesis components, for example a prosthetic knee joint and/or a lower-leg tube. On the right-hand side of FIG. 9, the tubular textile layer 40 is shown, with the seam allowance 44 and the turn-back region 48, which is formed circumferentially or almost circumferentially around the entire upper edge of the tubular envelope.

The final assembly or joining of the prosthesis cosmetic is shown schematically in FIG. 10. The base body 10 is provided in the final, desired shape, for example worked from a basic shape, for example cut and/or ground. The base body 10 is enveloped by the textile or the textile layer 40 on its outer side and is covered by the turned back region or the turn-back region 48 also at the upper end edge and the upper edge region of the base body 10 on the inner side of the cavity. In the exemplary embodiment shown, the textile layer 40 is pulled over the base body 10 from above. In the exemplary embodiment, the base body 10 has, on the rear side, a groove or a slot along which the seam 43 or the seam overhang 44 is guided. This ensures easier alignment of the textile layer 40 relative to the base body 10 during the pulling-on or the relative movement with respect to each other. This also ensures that the shape of the upper edge of the base body 10 matches the shape and the pocket 48 at the upper end of the textile layer 40. If the textile layer 40 is correctly pulled onto the base body 10, the seam allowance 44 lies perpendicular to the rear edge of the base body 10, with the seam allowance 44 protruding outward. After the textile layer 40 has been pulled on, the adhesive layer can be heated and activated. It is likewise possible for an already heated base body 10 and/or a heated textile layer 40 to be provided and used and for them to be arranged on each other as described above.

If the base body 10 has not been adequately or completely bonded to the textile layer 40, the required thermal energy can be added subsequently, for example using an iron or a hot air blower. The textile layer 40 is then fixed to the base body 10 under pressure. Glue can likewise be applied subsequently to the non-glued regions in order to achieve the attachment by suitable subsequent measures.

If there is a textile overhang at the distal, lower end, this can be removed. Alternatively, the protruding region can be heated and turned inward, or vice versa, in order to be glued to the base body 10 at the lower edge.

As an alternative to the arrangement at the proximal end or in addition to an arrangement there, the turn-back region 48 or the pocket can also be arranged or formed at the distal end.

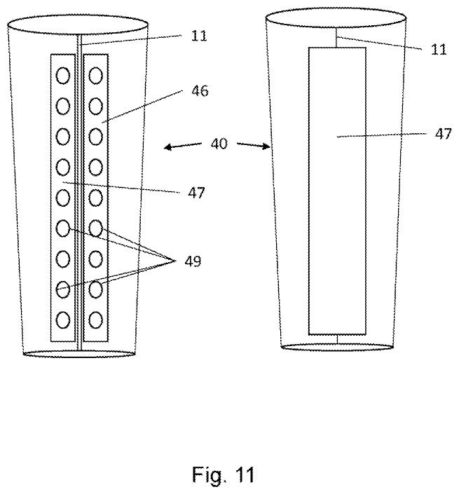

FIG. 11 shows a further variant of the textile 40. Both the textile 40 and the prosthesis cosmetic, with a base body (not shown in more detail) arranged therein or surrounded thereby, are shown in an opened state in the illustration on the left. In the illustration on the right, the prosthesis cosmetic is shown in a closed state. Two fastener elements 46, 47 are arranged or fastened along the slit 11 within the base body, for example in the region of the seam allowance 44 or at the outer regions oriented along the seam 43 next to the slit 11. The fastener elements 46, 47 are arranged on the textile 40, for example sewn, welded or glued on. In the exemplary embodiment shown, the fastener elements 46, 47 are equipped with a multiplicity of correspondingly arranged magnets 49 which attract each other in the closed state. Instead of two magnets 49, a ferromagnetic element can also be arranged on the respective other side of a magnet 49. In the exemplary embodiment shown, the left-hand form-fit element 47 is designed to be foldable, while the right-hand form-fit element 46 is fixed on the outer side of the textile 40. In the illustration on the left in FIG. 11, the slit 11 or gap is opened, and the fastener elements 46, 47 are not in engagement with each other. In the illustration on the right in FIG. 11, the two end faces (not shown in more detail) of the base body are moved toward each other, bear on each other or are in maximum approximation to each other. To close the slit 11, the left-hand fastener element 47 is folded over and placed on the right-hand fastener element 46 and fixed there magnetically by frictional connection. In this way, the circumference in the region of the fastener elements 46, 47 is closed and the prosthesis cosmetic is fixed.

1. A prosthesis cosmetic with a base body made of a foam material and/or fiber composite material and with a cavity formed in the base body for receiving a prosthesis component, the base body having an outer side which faces away from the cavity and on which a textile layer is arranged, wherein an adhesive is applied to the inner side of the textile layer facing the base body, and the textile layer is adhesively bonded or laminated to the outer side of the base body.

2. The prosthesis cosmetic of claim 1, wherein the adhesive is thermally activatable.

3. The prosthesis cosmetic of claim 1, wherein the textile layer is formed from a tube or a flat blank.

4. The prosthesis cosmetic of claim 1, wherein the textile layer and/or the base body are composed of a flexible, and/or in particular an elastic material.

5. The prosthesis cosmetic of claim 1, wherein the textile layer and/or the base body are composed of an already pre-shaped, and/or in particular a non-elastic material.

6. The prosthesis cosmetic of claim 1, wherein the base body has a slit with two mutually opposite end faces.

7. The prosthesis cosmetic of claim 6, wherein the textile layer is adhesively bonded to the end faces.

8. The prosthesis cosmetic of claim 6, wherein a fastener is arranged on the textile layer, via which fastener the end faces are coupled to each other in a closed state of the fastener.

9. The prosthesis cosmetic of claim 1, wherein the outer side of the textile layer is provided with a water-repellent, flame-retardant and/or antimicrobial coating and/or finish.

10. The prosthesis cosmetic of claim 1, wherein a pocket or a turn-back region, into which the base body is inserted, is formed at an upper and/or lower edge of the textile layer.

11. A method for producing a prosthesis cosmetic of claim 1, having the following steps:
making available a base body made of a foam material and/or fiber composite material and adapted to a prosthesis component to be covered and to a prosthesis wearer,
applying an adhesive to the inner side of a textile layer facing the base body, and
arranging and gluing the textile layer on the outer side facing away from the prosthesis component when the prosthesis cosmetic is in place.

12. The method of claim 11, wherein the adhesive is thermally activatable, and the textile layer is glued on with application of pressure and heat.

13. The method of claim 11, wherein the textile layer is wrapped or sheathed with a textile or a film before and during the delivery of heat.

14. The method of claim 11, wherein the adhesive layer is applied at least to regions of the inner side of the textile layer.

15. The method of claim 14, wherein, on the inner side, the adhesive layer is applied over the whole surface or over the entire contact surface on the base body in regions, and/or in particular at points.

16. The method of claim 11, wherein the textile layer is designed as a one-piece tube or as a tube formed from a blank via a seam and, before gluing, is pulled over the base body.

17. The method of claim 11, wherein the base body is ground before being connected to the textile layer.

18. The method of claim 11, wherein the textile layer is separated along a slit formed in the base body and is provided with a fastener.

19. The method of claim 18, wherein the textile layer is cohesively connected to the base body on mutually opposite end faces of the slit.

20. A system composed of a base body and of a textile layer coated with a thermally activatable adhesive, as claimed in claim 1.
